(19) United States
(12) Patent Application Publication
(10) Pub. No.: US 2004/0073192 A1
(43) Pub. Date: Apr. 15, 2004

(54) Intravenous fluid delivery set

(76) Inventors: Mary Jane Flament-Garcia, Wadsworth, IL (US); Michael D. Laferriere, Gurnee, IL (US); Natwar S. Patel, Vernon Hills, IL (US); Erin Kim Pavlovic, Zion, IL (US); Paul J. Somodi, Lindenhurst, IL (US); Derwin S. Threats, Waukegan, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/270,819

(22) Filed: Oct. 15, 2002

(51) Int. Cl. A61M 25/00

(52) U.S. Cl. 604/523; 604/257

(57) ABSTRACT

A flexible tubing for an intravenous fluid delivery set including an elongate tubing member formed of aliphatic polyether polyurethane. The set may include connectors to a container and an intravenous needle, and additional connectors for components placed in line with the tubing, with hollow posts on the connectors, with the tubing ends adapted to friction fit over the posts. The posts may have outer diameters which are tapered from a first diameter to a second diameter, where the inner diameter of the tubing ends are less than the second diameter. A method of delivering therapeutic fluid is also shown including providing the delivery set, providing a flow path by forcing tubing ends over connector posts in an in-line arrangement, and then controlling the flow of the therapeutic fluid between the container and the intravenous needle through the fluid flow path.

### BACKGROUND OF THE INVENTION

The present invention is directed toward intravenous fluid delivery sets.

Intravenous fluid delivery sets (“IV sets”) are well known in the art which contain a variety of components which can be combined, as desired for a particular patient, to facilitate the delivery of fluids directly to a patient's bloodstream. Such IV sets are well known for use in connecting a container such as a bottle or bag containing a liquid (e.g., a saline solution) to an intravenous needle extending into a patient's vein and suitably secured to the patient at the entry point (e.g., taped to the back of the patient's hand).

The IV sets typically include a tubing extending between hood assemblies, one of which is suitably connected to the container of liquid and the other of which is connected to the intravenous needle secured to the patient. Other components may also be provided for mounting at intermediate locations along the tubing, typically at breaks in the tubing between adjacent in-line tubing sections. Components in addition to the tubing and hood assemblies can include, for example, adapter, filters, clamps for selectively closing the tubing, back check valves, flow control devices for controlling the rate of flow of the liquid through the tubing and into the patient, needle-less connectors allowing for easy injection of a desired drug directly into the flow through the IV set, sight chambers allowing visual verification of the drip flow rate through the IV set, etc. IV sets which are not expected to rely upon gravity for flow may also include pump structures as well.

Among the desirable features of IV sets are the ability to easily and reliably set up the IV set in a variety of different conditions (sometimes in time critical situations) and the ability to easily and reliable deliver the desired fluids, and control the delivery of those fluids, for periods of time thereafter.

Heretofore, the components of IV sets have commonly been manufactured in large part with polyvinyl chloride (PVC). PVC tubings have been easily sealed to the other components using suitable solvents allowing easy bonding to a wide variety of materials, including acrylics, polycarbonates, and vinyls. However, some customers for IV sets have expressed a desire to avoid the use of PVC for a variety of reasons. It is, of course, desirable to provide customers with a product which meets their requirements.

Materials other than PVC have been heretofore used with IV sets. For example, polyurethane tubings have been used in prior art IV sets. However, those IV sets have to date proven undesirable for a number of reasons, including an insufficient resistance to kinking of the tubings and an insufficient maintenance of clarity of the material. Of course, inadvertent kinking of a tubing could cause the flow of fluid to be cut off, with potentially disastrous results, and is a serious concern given the bending to which IV sets are commonly subjected (e.g., due to movement of the patient). Resistance to kinking requires an element of stiffness for the tubing, which can be difficult to balance with the contradictory requirement of flexibility for the tubing. Further, some tubings used in the market and formed of aromatic non-PVC materials can color during gamma radiation sterilization, for example, leaving a poor appearance after such handling. Clouding and/or coloring of the tubings can not only inhibit the desired ability to view inside the tubings to help verify proper flow, but can cause patients to incur increased stress if they interpret an unclear tubing to indicate a lack of sterility of the IV set being used to deliver fluids to their bloodstream.

The present invention is directed toward overcoming one or more of the problems set forth above.

### SUMMARY OF THE INVENTION

In one aspect of the present invention, a flexible tubing for an intravenous fluid delivery set is provided, including an elongate tubing member formed of aliphatic polyether polyurethane.

In one form of this aspect of the invention, the aliphatic polyether polyurethane is a polymer of dicylohexylmethanediisocyanate, poly(tetramethylene glycol) and 1,4-Butanediol.

In another aspect of the present invention, an intravenous fluid delivery set is provided, including a first connector for connecting to a container, and an aliphatic polyether polyurethane elongated hollow tubing having open distal and proximate ends, at least one of which is adapted for a fluid tight connection to the connector.

In another form of this aspect of the invention, the one or more connectors and the tubing define a fluid delivery path free of polyvinyl chloride.

In still another form of this aspect of the invention, the connector is a female connector that each includes a cylindrical recess adapted to securely receive one of the proximate or distal tubing ends therein.

In an alternate form of this aspect of the invention, the connector has a hollow post and the tubing end has an inner diameter less than the post outer diameter and is adapted for friction fit over the male connector post.

In another form of this alternate form including a connector post, the hollow post has an outer diameter that is tapered from a first diameter to a second diameter, and the elongated hollow tubing distal or proximate end inner diameter is less than the second diameter.

In still another form of the alternate form including a connector post, the tubing has an inside diameter on the order of 0.095″ and an outside diameter on the order of 0.150″. In a further form, the outer diameter of the hollow post tapers from a first outer diameter at the end of the post to a second outer diameter on the order of 0.134″, the first outer diameter being less than 0.134″.

In yet another form of this aspect of the invention, multiple connectors and tubings are provided utilizing over the post friction fits.

In yet another aspect of the present invention, a method of delivering therapeutic fluid is provided, including providing an intravenous fluid delivery set, providing a fluid flow path between a therapeutic fluid container and a patient using the delivery set, and controlling the flow of the therapeutic fluid between the container and an intravenous needle or catheter through the fluid flow path. The provided delivery set includes first and second connectors, at least one set component chosen from the group of filters, valves, flow control devices, needle-less connectors and pumps, and first and second aliphatic polyether polyurethane hollow tubings. The flow path is provided by (1) connecting the first connector to a container of therapeutic fluid, (2) connecting the second connector to an intravenous needle in a patient, (3) connecting the proximate end of the first tubing to the first connector and connecting the distal end to the third connector of one of the at least one component, and (4) connecting the distal end of the second tubing to the second connector and connecting the proximate end to the fourth connector of one of the at least one component.

In another form of this aspect of the invention, the step of providing an intravenous fluid delivery set comprises providing connectors which each have a cylindrical recess adapted to securely receive the proximate and distal tubing ends therein.

In an alternate form of this aspect of the invention, the step of providing an intravenous fluid delivery set includes (1) providing the connectors each with a hollow post with a selected outer diameter and (2) providing the tubings with the open ends having inner diameters less than the selected outer diameter, where the tubing connecting steps comprise forcing the tubing ends over the hollow posts. With this alternate form, the selected outer diameter may be tapered from a first diameter to a second diameter, with the inner diameter of the tubing ends being less than the second diameter. With this alternate form, the first and second tubings may having an inside diameter on the order of 0.095″ and an outside diameter on the order of 0.150″, and the selected outer diameter may taper from a first outer diameter at the ends of the posts to a second outer diameter on the order of 0.134″, said first outer diameter being less than 0.134″.

### BRIEF DESCRIPTION OF THE DRAWINGS

FIG. 1 is a partially exploded perspective view illustrating the use of a fluid delivery set embodying the present invention;

FIG. 2 is an exploded view showing the connection of a tubing and connector according to the present invention;

FIG. 3 is an exploded view showing an alternative connection of a tubing and connector according to the present invention;

FIG. 4 is a plan view showing another arrangement of a fluid delivery set embodying the present invention; and

FIG. 5 illustrates the chemical composition of an aliphatic polyether polyurethane from which tubing of the present invention may be made.

### DESCRIPTION OF THE PREFERRED EMBODIMENT

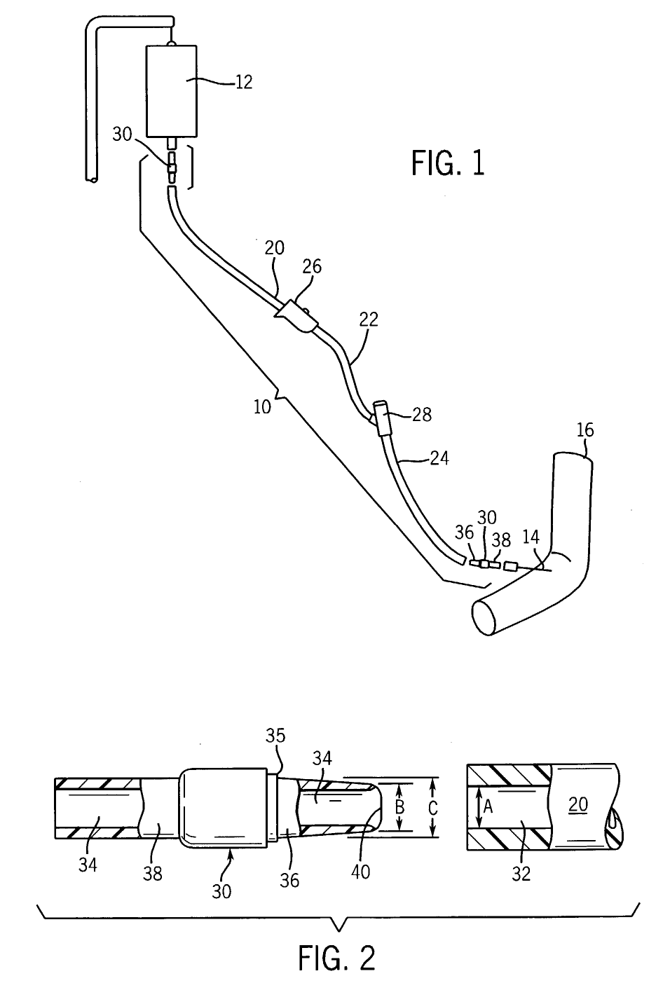

An assembled intravenous fluid delivery set 10 embodying the present invention is illustrated in FIG. 1. The set 10 may be particularly adapted for delivering a fluid from a container 12 (e.g., a bottle or bag) to an intravenous needle 14 secured in a vein of a patient 16. It should be understood, however, that sets embodying the present invention can be used in a variety of applications, including neonatal tubing sets and blood tubing sets. In applications where fluid (e.g., blood) is extracted from a patient, the container 12 may be a suitable receptacle for the fluid, whereas when therapeutic fluid is being provided to the patient, the container 12 dispenses the fluid (e.g., a saline solution and/or prescribed drugs).

In the particular application illustrated in FIG. 1, various parts of the fluid delivery set 10 are illustrated in an in-line arrangement providing a fluid path between the container 12 and intravenous needle 14, including three tubings 20, 22, 24, a roller clamp 26 and an integral needle-less connector 28. However, it should be understood that while FIG. 1 shows a set 10 in use with the particular illustrated components, the set 10 may be provided in kit form with additional components such as previously described (e.g., filters, back check valves, sight chambers, etc.), which components may or may not be used in a particular instance due to the conditions of the particular use. Preferably the above-mentioned additional components are free of polyvinyl chloride (PVC) so as to define a fluid delivery path free of PVC. For example, a polycarbonate (non PVC) sight chamber is available from Borla S.p.A. of Torino, Italy.

FIG. 2 illustrates a connector 30 and an end of a tubing (identified as tubing 20 for illustration purposes). Both the tubing 20 and connector 30 include openings 32, 34 therethrough which, when connected, cooperate to define the desired fluid path. The connector 30 includes a hollow post 36 over which the end of the tubing 20 is friction fit (i.e., secured by mechanical interference, not chemical bonding). Before forcing the tubing end over the post 36, a suitable solvent (e.g., a cyclohexane and methylene chloride blend) may be placed on the post 36, which solvent acts as a lubricant as the tubing 20 slides over the outer surface of the post 36.

While reference herein is made to a connector 30 such as shown in FIGS. 1 and 2 in which a discrete structure preferably of a semi-rigid to substantially rigid non PVC material including but not limited to acrylic, acrylonitrile butadiene styrene (ABS), or polycarbonate is provided, the discussion of the connectors 30 (with hollow posts 36) herein should be recognized as pertaining to a wide variety of connecting structures. For example, the connector could comprise one or more hollow posts integral to a component (e.g., roller clamp 26 and integral needle-less connector 28) allowing direct connection of tubings to those components, or the connector could comprise a discrete adapter structure which includes a hollow post for connecting to a tubing and an adapter for connecting to a component. That is, the connector could be a hollow post integrally provided on an intravenous needle, or could be a separate adapter structure (such as illustrated in FIG. 1) which includes a hollow post 36 for securing to a tubing 24 with a hollow adapter 38 configured to attach to the attachment structure of the intravenous needle 14. The adapter 38 preferably has a hollow tapered post configuration similar to that described below for the hollow post 36.

It should also be understood that, as used herein, “connection” of a tubing to some other element may not be a direct connection between the tubing end and that element, but may comprise the disclosed connection of the tubing to a connector which is itself otherwise, directly or indirectly, suitably connected to that element.

As shown in FIG. 2, the hollow post 36 has an outer diameter which is greater than the inner diameter of the tubing 20. More specifically, the tubing 20 has an inner diameter A and the outer diameter of the post 36 tapers from B to C, where A is less than C. Such a configuration requires that the tubing 20 stretch as it is forced over the post 36, thereby providing a tight securement around the post 36 and a suitable friction force between the surfaces preventing the tubing 20 from being pulled off the post 36.

The end of the post 36 may also include a rounded tip 40 to further ease the end of the tubing 20 over the post 36, where the rounded tip 40 at its end has a diameter which is preferably no more than the inner diameter A of the tubing 20.

FIG. 3 illustrates another connector 42 and an end of a tubing (identified as tubing 20 for illustration purposes) which may be used within the broad scope of the invention. Both the tubing 20 and connector 42 include openings 32, 44 therethrough which, when connected, cooperate to define the desired fluid path. The connector 30 includes a recessed cylindrical opening 46 into which the end of the tubing 20 is secured. While some friction fit may occur, with this connector 42 it is preferred that a suitable conventional adhesive also be used to provide a suitable secure bond between the tubing 20 and the connector 42. The outer portion 48 of the recessed cylindrical opening 46 may be tapered to assist in guiding the end of the tubing 20 into the opening 46.

Still other connectors could be used within the scope of the invention, including securing the annular end face of the tubing to a surface surrounding an opening to the fluid path. While such a connection would not generally be as preferred as those illustrated in FIGS. 2-3, with the use of suitably secure adhesive it could still be used within the broad scope of the present invention.

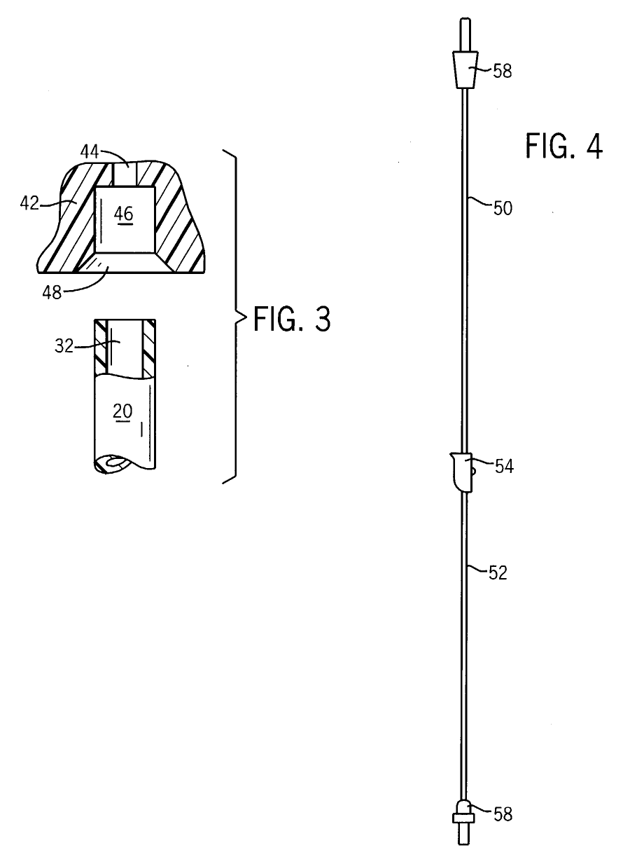

FIG. 4 simply illustrates another application of the fluid delivery set 10 of the present invention, with two tubings 50, 52 being secured in accordance with the above description to opposite connectors of a roller clamp 54, with hood adapter assemblies 56, 58 similarly secured to the other ends of the tubings 50, 52. The hood adapter assemblies 56, 58 may be particularly suited for directly connecting to, for example, a fluid container and intravenous needle.

In accordance with the present invention, the tubings are made of aliphatic polyether polyurethane.

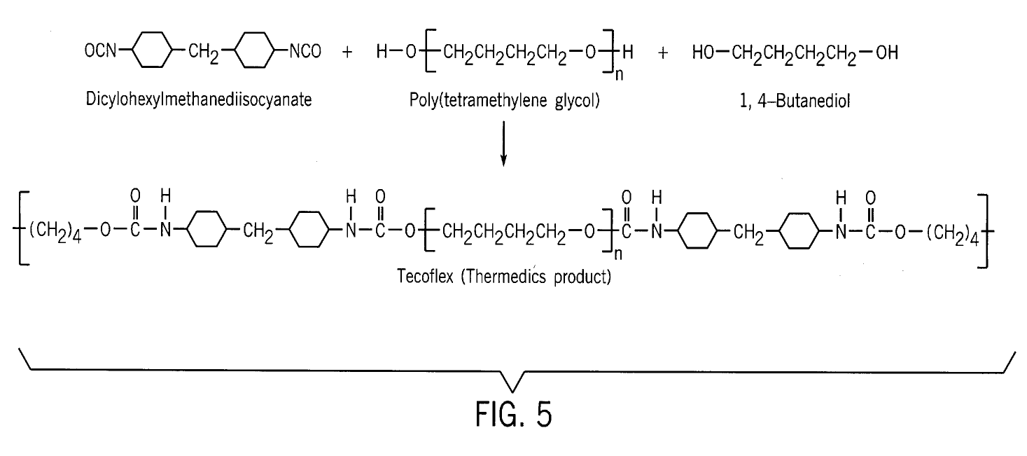

More specifically, in a preferred form, the tubings (e.g., 20, 22, 24) used in accordance with the present invention may be made of extruded polyurethane made from a non-aromatic, ether based, aliphatic polymer resin, such as that commercially available under the trade designation Tecoflex EG 85A from Thermedics, Inc. of Woburn, Mass. (Thermedics is the owner of U.S. Pat. Nos. 4,523,005 and 4,447,590, the complete disclosures of which are hereby incorporated by reference). FIG. 5 hereof illustrates such a polymer including the components from which it may be made. Specifically, the polyurethane is a polymer of dicylohexylmethanediisocyanate, poly(tetramethylene glycol), and 1,4-Butanediol. The preferred durometer hardness for the tubing is about 77 Shore A.

In accordance with the present invention, the material may also be free of diethyl hexyl phthalate (DEHP), which is a plasticizer which can undesirably cause extraction and reduced potency with some drugs (e.g., nitroglycerine).

The above described aliphatic polyether polyurethane material avoids clouding or discoloration such has occurred, for example, when prior art polyurethane tubings were sterilized.

While the size of the tubings could vary according to particular needs, a preferred form made with the above described polyurethane has an inside diameter (A) on the order of 0.095″ and an outside diameter on the order of 0.150″ (providing a nominal wall thickness of 0.0275″ and a ratio of wall thickness divided by inside diameter of about 0.29). The hollow posts 36 of the components such as the connector 30 illustrated in FIG. 2 may then have an outer diameter which includes a rounded end beginning at the very end at a diameter of at least about 0.095″ to an outer diameter (B) of about 0.119″, with the post 36 then relatively smoothly tapering to an outer diameter (C) of about 0.134″. Such a structure allows the end of a tubing (e.g., 20, 22, 24) to be relatively easily pushed over the end of the post 36 while at the same time thereafter providing a strong friction securement therebetween. A shoulder 35 may be provided on the conncector to positively limit the position of the end of the tubing (e.g., 20,22,24). Such fittings have, for example, been found to have a tubing pull off force greater than eight pounds with an effective bond strength greater than or equal to PVC solvent sealed bonds used in the prior art, while also providing required kink resistance and allowing easy connection between tubing and post.

Another preferred form, usable in neonatal applications, made with the above described polyurethane has an inside diameter (A) on the order of 0.054″ and an outside diameter on the order of 0.084″ (providing a nominal wall thickness of 0.015″ and a ratio of wall thickness divided by inside diameter of about 0.28). Still another preferred form, usable for example in blood applications, made with the above described polyurethane has an inside diameter (A) on the order of 0.120″ and an outside diameter on the order of 0.170″ (providing a nominal wall thickness of 0.025″ and a ratio of wall thickness divided by inside diameter of about 0.21). Testing of such tubings has been found to provide an adequate pull off force when friction fit over posts in such applications.

Of course, variations on the mechanical interference fit described above could also be used in accordance with the invention. For example, a portion of the outer surface of the post 36 could be cylindrical rather than tapered, with the cylindrical portion having an outer diameter greater than the tubing inner diameter (A). As another example, small raised ribs could be provided on the outer diameter of the posts.

Tests have been performed to evaluate tubings and connectors in accord with the above structure against prior art tubings made with PVC. The below table illustrates the results of this testing:

| MECHANICAL PROPERTIES | PVC | PU |
|---|---|---|
| Average Values/Material | | |
| Tensile Strength (psi) | 2900 | 4400 |
| Ultimate Elongation (%) | 400 | 1300 |
| 100% Modulus (psi) | 1600 | 700 |
| KINK RESISTANCE (dia.-inches) | 0.500 | 0.560 |
| COMPRESSION TESTING (ml/min) | | |
| Roller Clamp | | |
| initial | 243.3 | 185.9 |
| 10 minutes | 243.6 | 188.2 |
| 6 hours | 238.0 | 189.54 |
| 24 hours | 230.3 | 194.2 |
| initial | 233.5 | 194.1 |
| 10 minutes | 233.7 | 193.0 |
| 6 hours | 235.1 | 196.9 |
| 24 hours | 226.8 | 198.1 |

It should thus be appreciated that fluid delivery sets made according to the present invention may be provided at reasonable cost to customers who desire to avoid PVC materials (without incurring drawbacks such as undesirable coloring, kinking and/or compression) and such sets may be readily assembled for use in a variety of applications.

Still other aspects, objects, and advantages of the present invention can be obtained from a study of the specification, the drawings, and the appended claims. It should be understood, however, that the present invention could be used in alternate forms where less than all of the objects and advantages of the present invention and preferred embodiment as described above would be obtained.

1. A flexible tubing for an intravenous fluid delivery set, comprising an elongate tubing member formed of aliphatic polyether polyurethane.

2. The flexible tubing of claim 1, wherein the aliphatic polyether polyurethane is a polymer of dicylohexylmethanediisocyanate, poly(tetramethylene glycol), and 1,4-Butanediol.

3. The flexible tubing of claim 1, wherein said tubing member has an inside diameter on the order of 0.095″ and an outside diameter on the order of 0.150″.

4. The flexible tubing of claim 1, wherein said tubing member has a substantially uniform wall thickness, said wall thickness being in a range from about 0.015″ to about 0.0275″

5. The flexible tubing of claim 1, wherein said tubing member has a substantially uniform wall thickness and an inside diameter such that a ratio of the wall thickness divided by the inside diameter is about 0.21 to about 0.29.

6. An intravenous fluid delivery set, comprising:
a first connector; and
an elongated aliphatic polyether polyurethane hollow tubing having open distal and proximate ends, said proximate end adapted for a fluid tight connection to said first connector.

7. The intravenous fluid delivery set of claim 6, wherein the aliphatic polyether polyurethane is a polymer of dicylohexylmethanediisocyanate, poly(tetramethylene glycol), and 1,4-Butanediol.

8. The intravenous fluid delivery set of claim 6, wherein said first connector and said tubing define a fluid delivery path free of polyvinyl chloride.

9. The intravenous fluid delivery set of claim 6, wherein said first connector is a female connector that includes a cylindrical recess adapted to securely receive said proximate tubing end therein.

10. The intravenous fluid delivery set of claim 9, wherein a suitable adhesive is utilized to secure said proximate tubing end in said cylindrical recess of said first connector.

11. The intravenous fluid delivery set of claim 10, wherein said cylindrical recess of said first connector is annular.

12. The intravenous fluid delivery set of claim 6, wherein:
said first connector Is a male connector that includes a first hollow post with an outer diameter; and said elongated hollow tubing proximate end has an inner diameter less than said first hollow post outer diameter and is adapted for friction fit over said first hollow post.

13. The intravenous fluid delivery set of claim 12, wherein said outer diameter of said first hollow post is tapered from a first diameter to a second diameter, said inner diameter of said elongated hollow tubing proximate end being less than said diameter.

14. The intravenous fluid delivery set of claim 12, wherein said tubing has an inner or inside diameter on the order of 0.095″ and an outer or outside diameter on the order of 0.150″.

15. The intravenous fluid delivery set of claim 14, wherein said outer diameter of said first hollow post tapers from a first outer diameter of less than 0.134″ at a terminal end of said post to a second outer diameter on the order of 0.134″.

16. The intravenous fluid delivery set of claim 6, comprising:
a second connector adapted for a fluid tight connection to said distal end of said hollow tubing.

17. The intravenous fluid delivery set of claim 16, wherein said first and second connectors and said tubing define a fluid delivery path free of polyvinyl chloride.

18. The intravenous fluid delivery set of claim 16, wherein said first and second connectors are female connectors that include a cylindrical recess adapted to securely receive said proximate and distal tubing ends respectively therein.

19. The intravenous fluid delivery set of claim 6, wherein:
said first connector is a male connector that includes a first hollow post with an outer diameter;
said second connector is a male connector that includes a second hollow post with an outer diameter;
said elongated hollow tubing proximate end has an inner diameter less than said first hollow post outer diameter and is adapted for friction fit over said first hollow post; and
said elongated hollow tubing distal end has an inner diameter less than said second hollow post outer diameter and is adapted for friction fit over said second hollow post.

20. The intravenous fluid delivery set of claim 19, wherein said first and second hollow posts have outer diameters which are tapered from a first diameter to a second diameter, said elongated hollow tubing distal and proximate end inner diameters being less than said second diameter.

21. The intravenous fluid delivery set of claim 19, wherein said tubing has an inside diameter on the order of 0.095″ and an outside diameter on the order of 0.150″.

22. The intravenous fluid delivery set of claim 21, wherein the outer diameter of said first and second hollow posts tapers from a first outer diameter at a terminal end of said posts to a second outer diameter on the order of 0.134″, said first outer diameter being less than 0.134″.

23. The intravenous fluid delivery set of claim 19, further comprising:
a third hollow post on said second connector and in fluid communication with said second hollow post; and
a second aliphatic polyether polyurethane elongated hollow tubing having open distal and proximate ends,
said second tubing proximate end having an inner diameter less than said third hollow post outer diameter and adapted for friction fit over said third hollow post.

24. The intravenous fluid delivery set of claim 23, wherein said first, second and third hollow posts have outer diameters which are tapered from a first diameter to a second diameter, and said first tubing distal and proximate end inner diameters are less than said second diameter and said second tubing proximate end inner diameter is less than said second diameter.

25. The intravenous fluid delivery set of claim 23, wherein said second connector comprises a fluid control device.

26. The intravenous fluid delivery set of claim 23, wherein said second connector comprises a needle-less connector.

27. A method of delivering therapeutic fluid, comprising:
providing an intravenous fluid delivery set including:
a first connector,
a second connector,
at least one set component chosen from the group of filters, valves, flow control devices, needle-less connectors and pumps, said component including third and fourth connectors, and
first and second aliphatic polyether polyurethane elongated hollow tubings, each of said tubings having open distal and proximate ends;
providing a PVC-free fluid flow path between a therapeutic fluid container and a patient by
connecting said first connector to a container,
connecting said second connector to an intravenous needle,
connecting the proximate end of the first tubing to the first connector and connecting the distal end to the third connector of one of said at least one set component, and
connecting the distal end of the second tubing to the second connector and connecting the proximate end to the fourth connector of said one of said at least one set component; and
controlling the flow of said therapeutic fluid between said container and said intravenous needle through said fluid flow path.

28. The method of claim 27, wherein the aliphatic polyether polyurethane is a polymer of dicylohexylmethanediisocyanate, poly(tetramethylene glycol), and 1,4-Butanediol.

29. The method of claim 27, wherein said providing an intravenous fluid delivery set comprises providing connectors each having a cylindrical recess adapted to securely receive said proximate and distal tubing ends therein.

30. The method of claim 27, wherein:
said providing an intravenous fluid delivery set step includes
providing said connectors each including a hollow post with a selected outer diameter, and
providing said first and second elongated hollow tubings with said open distal and proximate ends having inner diameters less than said selected outer diameter; and
in said providing a fluid flow path step, said tubing connecting steps comprise forcing said tubing ends over said hollow posts.

31. The method of claim 30, wherein said selected outer diameter is tapered from a first diameter to a second diameter, said inner diameter of said tubing ends being less than said second diameter.

32. The method of claim 30, wherein said delivery set is provided with said first and second tubings having an inside diameter on the order of 0.095″ and an outside diameter on the order of 0.150″.

33. The method of claim 32, wherein said selected outer diameter tapers from a first outer diameter at the ends of said posts to a second outer diameter on the order of 0.134″, said first outer diameter being less than 0.134″.

34. The flexible tubing of claim 1, wherein said tubing member has an inside diameter on the order of 0.054″ and an outside diameter on the order of 0.084″.

35. The flexible tubing of claim 1, wherein said tubing member has an inside diameter on the order of 0.120″ and an outside diameter on the order of 0.170″.
